(19) United States
(12) Patent Application Publication
(10) Pub. No.: US 2024/0201091 A1
(43) Pub. Date: Jun. 20, 2024

(54) DOSIMETER MATERIAL FOR AMMONIA AND/OR AMINES, PRODUCTION AND USE OF SAME

(71) Applicants: Universitaet zu Luebeck, Luebeck (DE); Porphyrin-Laboratories GmbH, Scharbeutz (DE)

(72) Inventors: Ramtin Rahmanzadeh, Luebeck (DE); Gereon Huettmann, Luebeck (DE); Christian Schell, Stockelsdorf (DE)

(21) Appl. No.: 18/541,072

(22) Filed: Dec. 15, 2023

Related U.S. Application Data
(62) Division of application No. 17/263,011, filed on Jan. 25, 2021, now abandoned.

(30) Foreign Application Priority Data
Jul. 27, 2018 (DE) 10 2018 118 211.2

(51) Int. Cl. G01N 21/78; C07D 487/22; G01N 21/77; G01N 33/00; G01N 33/497

(52) U.S. Cl. CPC G01N 21/783; C07D 487/22; G01N 33/0054; G01N 33/497; G01N 2021/7786; G01N 2033/4975

(57) ABSTRACT

The invention relates to a dosimeter material for ammonia and/or amines, the indicator used, as well as processes for their manufacture and use, in particular for quality control of foodstuffs. The dosimeter material for ammonia and/or amines, in particular in the gas phase, comprises an indicator which undergoes an irreversible color change in the presence of ammonia and/or amines, and an immobilization matrix for the indicator that is permeable to ammonia and/or amines, wherein the immobilization matrix is water-impermeable, and wherein the indicator comprises a phosphorus porphyrin activated by covalent bonding to a silanol group and having the formula porphyrin-P(V)X₃, wherein X is Cl or Br.

The invention relates to a dosimeter material for ammonia and/or amines, the indicator used, as well as processes for its manufacture and use, in particular for quality control of foodstuffs.

In the food industry there is a great need to determine the shelf-life and freshness of food routinely and non-invasively. During the degradation of biological tissue, e.g. the spoilage of food derived from animals, protein degradation products such as amines are released into the environment in the gaseous phase. By detecting these, the freshness of food can be determined and a change in shelf-life (spoilage, ripeness) can be indicated. Up to now, amines can only be detected through complex laboratory analysis.

The analysis of amines in food chemistry/quality control/food monitoring is usually a complex process requiring high expenses for equipment, e.g., gas chromatography in combination with mass spectroscopy. Such techniques also require a complex and time- consuming sample preparation. Furthermore, trained personnel are required for operating the complex equipment. This altogether costly determination of the food condition leads to the fact that, in the production of food, a monitoring food control can only be carried out by sampling randomly. Similar problems arise in environmental analysis as well as in medical analysis. Especially in the food processing industry, there is an urgent need for time and cost-effective methods that allow a robust control of the shelf-life of the packaged product for each individual package.

The general idea of quality control in the gas phase of packaged food is known from the state of the art. For example, EP 0449798 A2 proposes a method for the quality control of packaged organic substances, in which the organic substance is enclosed together with an optical sensor element and thus brought into contact with the gas phase between the organic substance and the packaging, so that a change in the composition of the gas phase based on decomposition of the organic substance leads to a change in the color of the sensor element, which can be detected visually. This method is, amongst other things, also proposed for the detection of ammonia or amines. A more specific variant of this is the use of porphyrins in combination with a film as an optical sensor for amines. This is also known from the state of the art. For example, the utility model DE 212010000225 U1 describes a packaging material for determining the freshness of food, which consists of a sensor material and a film, whereby the detection of ammonia and amines released during the decomposition of fish or meat can occur by means of porphyrins.

Several tailor-made zinc-(II)- and chromium-(III)-metalloporphyrins have been used as chromophores for the colorimetric detection of amines (Heier, P. C. (2014) Novel metallo-porphyrin based colorimetric amine sensors and their processing via plasma enhanced chemical vapor deposition at atmospheric pressure synthesis, characterization and mechanistic studies. Dissertation, University of Mainz). For zinc-(II)-metalloporphyrin, a shift of the Soret band from 420 nm to 433 nm, and for chromium-(III)-metalloporphyrin, a shift of the Soret band from 435 nm to 452 nm is observed. The small absorption changes of the porphyrins used are unfavorable for use as amine sensors. Furthermore, chromium porphyrins are considered to be a health hazard, and therefore their use in connection with food is considered critical.

The objective of the invention is to provide a dosimeter material that can be adjusted to predetermined concentrations of ammonia and/or amines without cross-sensitivities to other substances, in particular to water, that is harmless to health and that can be evaluated visually, photometrically and/or fluorimetrically.

The problem is solved by the subject matter of the claims, in particular by a dosimeter material with the features of claim 3. As an intermediate product in the production of the dosimeter material according to the invention, the indicator according to claim 1 also is subject matter of the invention. The dependent claims relate to advantageous embodiments of the invention.

The dosimeter material for ammonia and/or amines according to the invention comprises an indicator that undergoes an irreversible color change in the presence of ammonia and/or amines, and an immobilization matrix for the indicator that is permeable to ammonia and/or amines, and wherein the immobilization matrix is water-impermeable.

The indicator of the invention comprises a phosphorus porphyrin activated by covalent bonding to a silanol group (also referred to as a silinol group), and having the formula porphyrin-P(V)X₃, wherein X is Cl or Br. The silanol group can be part of a coompound having a plurality of silanol groups and preferably a high surface area. For example, the indicator comprises a phosphorus porphyrin activated by covalent bonding to a silanol group of a compound comprising silica gel and having the formula porphyrin-P(V)X₃, wherein X is Cl or Br. Preferably, the indicator comprises a phosphorus porphyrin activated by covalent bonding to a silanol group of silica gel having the formula porphyrin-P(V)X₃, wherein X is Cl or Br.

In phosphorus porphyrins of the porphyrin-P(V)X₃ type, wherein X in particular comprises the halogens Cl or Br, two halogen ligands are axially bound to the phosphorus, and a halide counterion is responsible for charge balancing of the complex.

The indicator according to the invention can in particular comprise dibromo-phosphorus-(V)-tetraphenylporphyrin-bromide (TPP-P(V)Br₃), dichloro-phosphorus-(V)-tetratolylporphyrin-chloride (TTP-P(V)Cl₃), dichloro-phosphorus-(V)-2,3,7,8,12,13,17,18-octaethylporphyrin-chloride (OEP-P(V)Cl₃) or preferably dichloro-phosphorus-(V)-tetraphenylporphyrin-chloride (TPP-P(V)Cl₃).

A dosimeter material comprising an indicator according to the invention is particularly suitable for reacting with ammonia and/or amines in the gas phase.

The dosimeter material according to the invention is further characterized in that an activation of porphyrin-P(V)X₃ takes place by covalent bonding of one of the halogen ligands to a silanol group of surface-rich substances, in particular in silica gel.

According to the invention, a dosimeter material for ammonia and/or amines wherein the indicator comprises dichloro-phosphorus-(V)-tetraphenylporphyrin-chloride (TPP-P(V)Cl₃) activated by covalent bonding to silica gel is preferred.

The inventors have shown that TPP-P(V)Cl₃ activated by covalent bonding to silica gel surprisingly irreversibly reacts with ammonia and/or amines. This reaction leads to an irreversible color change of the TPP-P(V)Cl₃ indicator. This behavior is generally seen in the reaction of phosphorus porphyrin indicators with the formula porphyrin-P(V)X₃ with silanol groups. The reaction of the indicator with water also causes a color change of the indicator. Since the indicator is very sensitive to traces of moisture in the gas phase, it is necessary for the selective detection of ammonia and/or amines to avoid a possible reaction of the indicator with water. According to the invention, the indicator is introduced into a water-impermeable immobilization matrix for this purpose.

The phosphorus porphyrin indicators according to the invention, such as the TPP-P(V)Cl₃ indicator, react with ammonia and/or with amines in a 1:1 molar ratio. Since this reaction is irreversible, the irreversible color change can be interpreted integrally as an existing dose of ammonia and/or amines over time in the analyzed sample, especially in a gas mixture. The combination of phosphorus porphyrin indicator and water-impermeable immobilization matrix according to the invention can thus be used as a water-insensitive dosimeter for ammonia and/or amines.

According to the invention, the preferred color change is a change from green to red, which can be qualitatively evaluated with the naked eye, and in addition a clear wavelength shift in the absorption and fluorescence spectra—optionally changing the spectral shape—of the indicator material, which can be quantitatively (possibly automatically) recorded and evaluated.

The dosimeter material may be available as granules and/or film, preferably as a film, so that the samples that are to be investigated, which contain ammonia and/or amines can optimally react with the phosphorus porphyrin indicator, in particular with the TPP-P(V)Cl₃ indicator, in the dosimeter material.

The dosimeter material is further characterized in that the immobilization matrix comprises polymers impermeable to water and permeable to ammonia and/or amines, in particular polystyrene and/or preferably low-density polyethylene.

The process for preparing the phosphorus porphyrin indicator comprises an activation of porphyrin-P(V)X₃ by covalent bonding through a reaction of one of the halogen ligands of the porphyrin-P(V)X₃ with a silanol group of the surface-rich substance, preferably of silica gel.

This reaction takes place at elevated temperature, preferably between 80 and 140° C., preferably at 120° C., and preferably for 8 to 30 hours.

The process for preparing the TPP-P(V)Cl₃ indicator comprises an activation of the TPP-P(V)Cl₃ by covalent bonding to silica gel through the reaction of one of the chlorine ligands of the TPP-P(V)Cl₃ with a silanol group of the silica gel.

The process for preparing the dosimeter material comprises mixing the indicator with the immobilization matrix, wherein the process is carried out under exclusion of water, and wherein the mixture of indicator and immobilization matrix may preferably be present as granules and/or film.

The dosimeter material according to the invention can be used for the detection of ammonia and/or amines, wherein the color change of the indicator can preferably be detected visually. Fields of application are, for example, food quality control, medical applications such as respiratory gas analysis or wound healing dressings, or environmental analysis.

Further advantages, features and possible applications of the present invention can be seen from the following description in connection with the figures. The figures show:

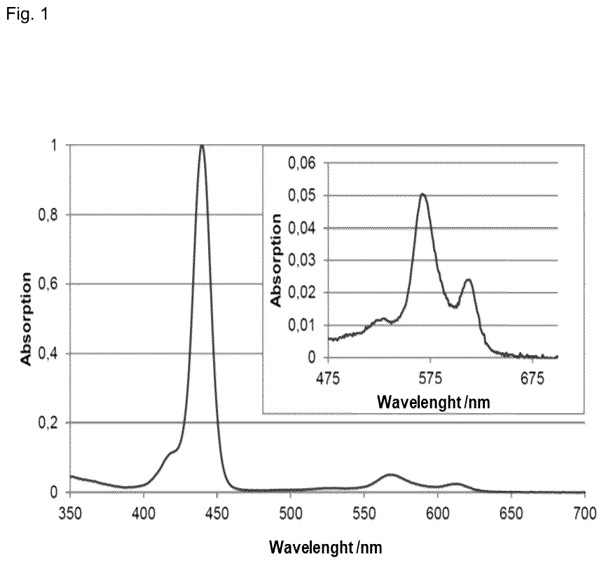

FIG. 1 UV/VIS spectrum of dichloro-phosphorus-(V)-tetraphenylporphyrin-chloride (TPP-P(V)Cl₃) in DCM.

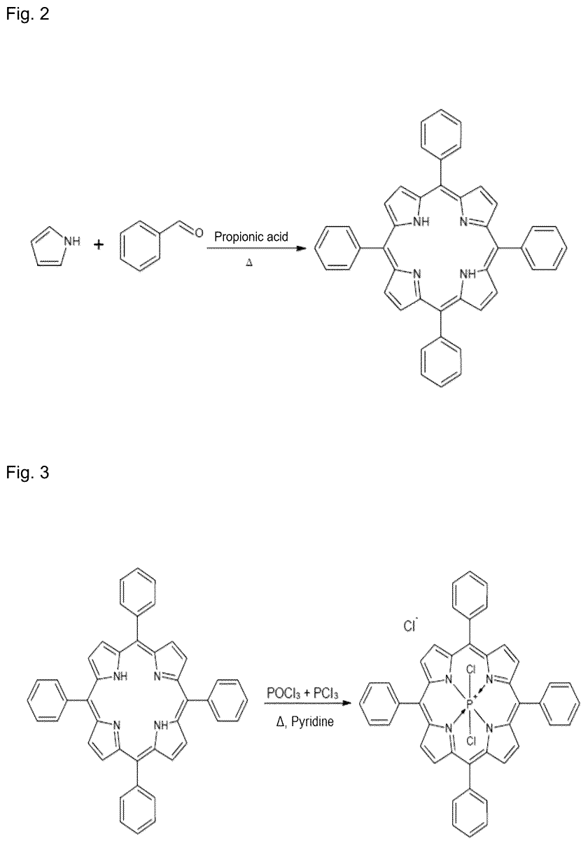

FIG. 2 Synthesis of tetraphenylporphyrin (TPP).

FIG. 3 Phosphorylation of TPP.

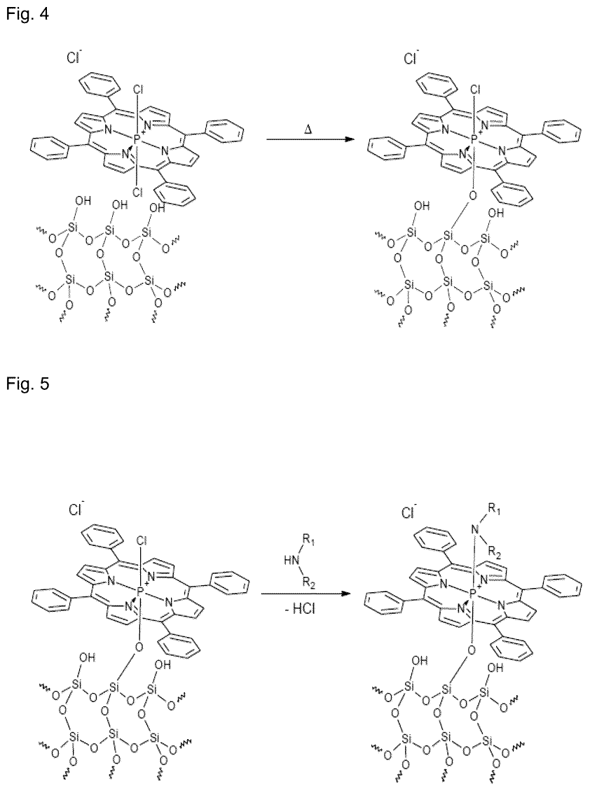

FIG. 4 Binding of TPP-P(V)Cl₃ to a silanol group on the surface of silica gel.

FIG. 5 Reaction of an amine with the activated chlorine ligand of TPP-P(V)Cl₃.

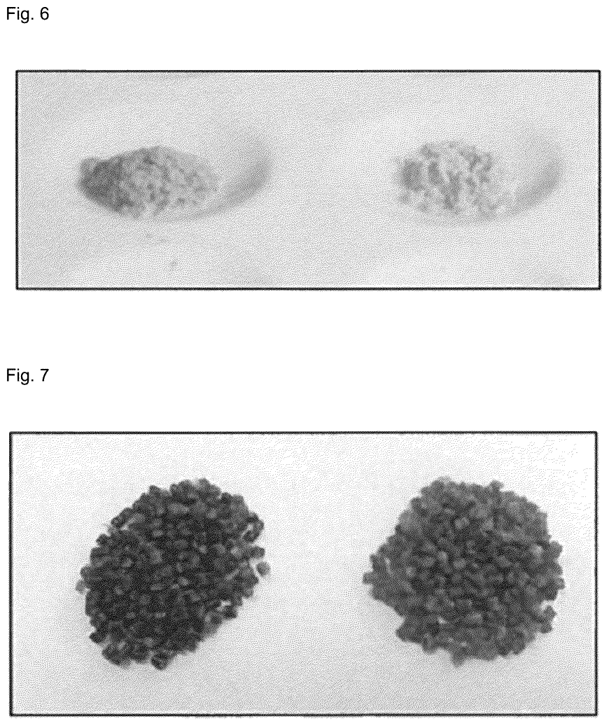

FIG. 6 Color change of the indicator powder: green in the absence of amines (left) and red after reaction with amines (right)

FIG. 7 Color change of the dosimeter material granules: green in the absence of amines (left) and red after reaction with amines (right).

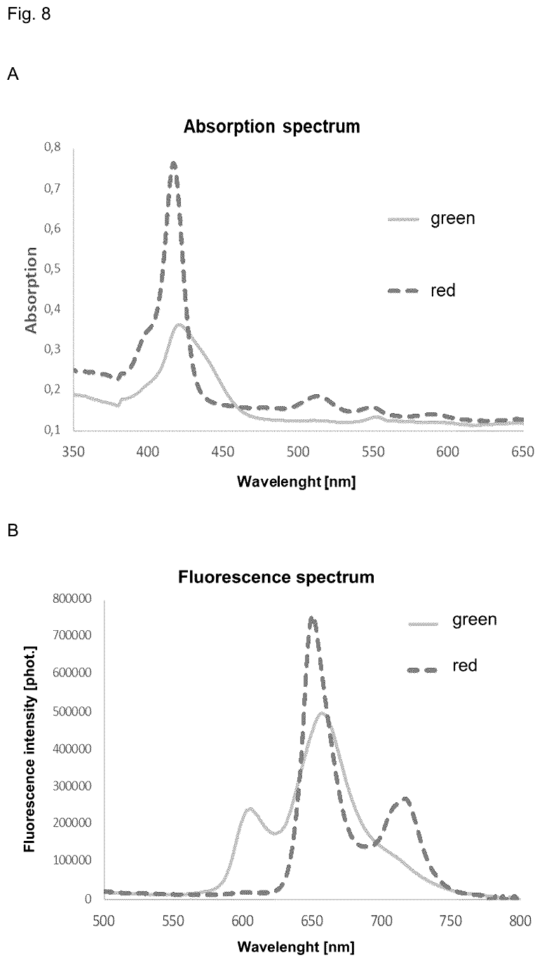

FIG. 8 Absorption (A) and fluorescence spectra (B) of the indicator before (green) and after (red) the reaction with amines.

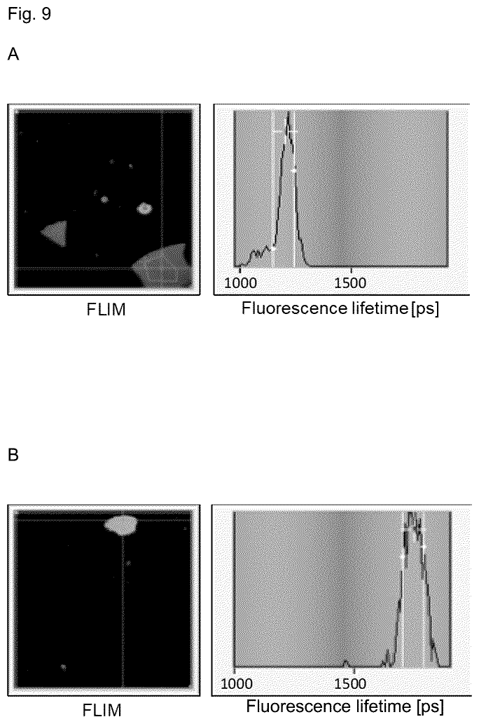

FIG. 9 Change in the fluorescence lifetime of the indicator: (A) green, (B) red.

The starting material for the preparation of the indicator for ammonia and/or amines according to the invention is a phosphorus porphyrin with the general formula porphyrin-P(V)X₃, for example dichloro-phosphorus-(V)-tetraphenylporphyrin-chloride (TPP-P(V)Cl₃). This is a porphyrin substituted with phenyl residues in meso position. In the aromatic ring system, pentavalent phosphorus having two axial chlorine ligands is coordinately bound. Charge balance is achieved via a chloride counterion. Phosphorus porphyrins, like other porphyrin complexes with elements of the fifth main group (arsenic, antimony and bismuth), have the special feature that they can occur in two different oxidation states. The rather unstable stage +III, also called hypervalent, and the stable oxidation stage +V. Among other things, these differ in their absorption spectrum. Phosphorus-(V)-porphyrins show a UV/VIS spectrum typical for porphyrins with one Soret band and two Q bands (FIG. 1).

In addition to the coordinative bond to the nitrogen atoms of the porphyrin ring and to the two axial ligands (halide, e.g. chloride), phosphorus-(V)-porphyrins carry a positive charge, which is compensated by a halide anion, e.g. a chloride anion. The axial halide ligands can be substituted by suitable nucleophiles. For example, the exchange against halogen, hydroxy, alkoxy or aryloxy groups is well known. However, it is known from the literature that in solution it is always both ligands that are substituted. In contrast, according to the invention, only one of the halide ligands of porphyrin-P(V)X₃, in particular only one of the chlorine ligands of TPP-P(V)Cl₃, is activated.

The preferred process for the production of the TPP-P(V)Cl₃ indicator according to the invention comprises the following steps:
a) Formation of tetraphenylporphyrin by reaction of pyrrole with benzaldehyde in boiling propionic acid: The TPP-P(V)Cl₃ is first produced in a two-step synthesis. For this purpose, pyrrole reacts with benzaldehyde in boiling propionic acid in a two-hour reaction (according to Adler et al. (1967) A Simplified Synthesis for meso-tetraphenylporphins. J. Org. Chem. 32 (2): 476) to form meso-tetraphenylporphyrin (TPP; FIG. 2). TPP crystallizes, with a yield of approx. 20%, on cooling. After filtering, washing with methanol and drying at approx. 120° C., the pure raw product can be phosphorylated.
b) Formation of TPP-P(V)Cl₃ from tetraphenylporphyrin by phosphorylation of the tetraphenylporphyrin by reaction with phosphorus trichloride and phosphoryl chloride in boiling pyridine: For this purpose, TPP reacts with an excess of a 1:1 mixture of phosphorus trichloride and phosphoryl chloride in boiling pyridine (FIG. 3).
c) Removal of the pyridine: The pyridine is preferably removed by distillation.
d) Purification of the TPP-P(V)Cl₃: This is preferably done by column chromatography using aluminum oxide.
e) Activation of TPP-P(V)Cl₃ by covalent bonding to silica gel through the reaction of one of the chlorine ligands of TPP-P(V)Cl₃ with a silanol group of the silica gel: To produce the active indicator, the green colored TPP-P(V)Cl₃ dissolved with in dichloromethane (DCM) is mixed with silica gel under exclusion of moisture, and the solvent is slowly evaporated in a rotary evaporator. Subsequently, a chlorine ligand of the phosphorus porphyrin reacts with a silanol group of the surface-rich silica gel or with a silanol group on the surface of nanoparticles, preferably at a temperature between 80 and 140° C., preferably at 120° C., in a drying oven for 8 to 30 hours (FIG. 4).

This special reaction activates the second chlorine ligand of TPP-P(V)Cl₃ (FIG. 5), so that it can very sensitively react with traces of ammonia and/or amines. This activation can lead to a reaction associated with a visually perceptible color change from green to red (FIG. 6), not only with ammonia and/or amines but also with water.

During the activation of phosphorus porphyrin, it is very likely that, due to steric hindrance, only one chlorine ligand will initially react with a silanol group of the silica gel in a solid phase reaction. As a result, the second chlorine ligand is extremely activated and a surprisingly irreversible reaction with water as well as with ammonia and/or amines can take place.

In U.S. Pat. No. 7,772,215 “Water detection composition and water detection indicator”, a phosphorus porphyrin with two axial chlorine ligands is also initially produced. By a further synthesis, the chlorine ligands are exchanged for hydroxy ligands before the new phosphorus complex is adsorbed on silica gel in the presence of calcium chloride. Then the silica gel is dried at 100° C., and it can detect moisture (anhydrous silica gel is green; wet silica gel is red). Here, this process can be reversed by drying the silica gel by heating, and is therefore reversible. The absorption of the hydroxy-ligand-containing phosphorus porphyrin complex is an electrostatic interaction with the silanol groups of the silica gel. Due to the hydroxy ligands, no covalent bonding with the silanol groups can occur. In contrast to U.S. Pat. No. 7,772,215, in the present invention the silanol groups of the silica gel react with the central phosphorus atom to form a covalent bond, since here, the phosphorus complex with the chlorine ligands is brought to chemically react with the silanol groups of the silica gel at elevated temperature, preferably between 80 and 140° C., and preferably for 8 to 30 hours. Although the silica gel-porphyrin complex of the present invention also shows a color change from green to red with water, no color change from red to green takes place during drying (e.g. by heating). The decisive difference to U.S. Pat. No. 7,772,215 therefore is that the water-induced color change of the indicator according to the invention is irreversible. The reason for this is that, in the present invention, the phosphorus porphyrin is covalently bound to a silanol group of the silica gel, and only the second chlorine ligand still present reacts with water.

For a reliable detection of ammonia and/or amines with the dosimeter material according to the invention, the side reaction of the indicator with water, which also causes a color change from green to red and is extremely sensitive to traces of moisture in the gas phase, must be prevented. After the indicator, e.g. the TPP-P(V)Cl₃ indicator, has been prepared, it must therefore be protected from traces of moisture. At the same time, the diffusion of ammonia and/or amines and their contact with the active indicator must not be prevented. By embedding the active indicator in a semi-permeable matrix, i.e. an immobilization matrix that is permeable to ammonia and/or amines and impermeable to water, the cross-sensitivity to water is eliminated. The decisive factor in the present invention thus is the embedding of the moisture-sensitive indicator in an immobilization matrix, preferably a polymer matrix. Herein, the amine-permeable polymer not only functions as a carrier/immobilization matrix for the indicator, but also prevents a color change of the indicator caused by moisture, and thus is an essential component in the function of the dosimeter material according to the invention.

Polymers that do not exhibit any permeability to water (including water vapor), but that are permeable to ammonia and/or amines are suitable as an immobilization matrix. Low-density polyethylene (LDPE; density between 0.910 and 0.940 g/cm³) is particularly suitable. Polymers such as polystyrene (PS) are also suitable as an immobilization matrix. Furthermore, a polymer mixture is conceivable as long as such a multi-component system has the physical properties with respect to gas diffusion and water absorption required for an immobilization matrix according to the invention.

To produce the PS-based dosimeter material, the indicator is stirred into a highly viscous solution of polystyrene in toluene, and then poured into thin layers of about 1-2 mm thickness. After the toluene evaporates, a highly active film is formed. A disadvantage of this production process, however, is the possible process-related solvent residue in the film, that could contaminate the foodstuffs packed in it. The potential toxic load can be avoided by using a film produced by thermal extrusion.

A preferred alternative to this manufacturing process therefore is a thermal mixing of the indicator with the highly hydrophobic polymer LDPE that has a good permeability for ammonia and amines. LDPE is also known from the state of the art for an extremely low water absorption, at the same time it has a high permeability for nitrogen, oxygen, carbon dioxide, as well as many odorous and aromatic substances. The green indicator powder is thermally distributed in the polymer by extrusion. LDPE has the advantage of a low processing temperature of 160-220° C. In this process a green granulate is produced. The presence of amines causes the granules to change color from green to red (FIG. 7). The green granules can then be processed into a film that also changes color from green to red in the presence of amines.

The indicator can be added to the immobilization matrix in any amount. Preferably, the indicator is added to the immobilization matrix in an amount just sufficient to give the immobilization matrix sufficient coloration to be visible to the naked eye. According to a particularly preferred embodiment, the indicator is added to the immobilization matrix in an amount of 0.1 to 5.0% (w/w), based on the total amount of immobilization matrix.

With light-scattering additives added to the phosphorus porphyrin in different ratios an increase in sensitivity by increasing the optical contrast is mad possible. Especially titanium oxide, which is known as a white pigment from the color industry, leads to a better visibility of the visually and colorimetrically detectable color change from green to red.

The problem of water cross-sensitivity of the indicator is solved by embedding it in the immobilization matrix: Even after several weeks of immersion in water, no moisture-related color change can be observed in LDPE dosimeter films.

The dosimeter material according to the invention exhibits selectivity for ammonia and/or amines. It shows a particularly good response to amines with a molar mass of less than 150 g/mol. Examples of amines within the scope of the present invention are diethylamine, trimethylamine, triethylamine, ethanolamine, hexylamine, cadaverine and putrescine. In cross-sensitivity tests, no color change of the dosimeter material with thiols, amino acids, alcohols, aldehydes or ketones was observed (see example 6).

The dosimeter material according to the invention has a high sensitivity for ammonia and/or amines. A colorimetrically detectable color change occurs with a sensor area of one square centimeter and a film thickness of 100 μm in the range of at least 20 nmol. This sensitivity can be improved by at least a factor of 100 by metrological evaluation, especially of fluorescence properties.

The marked change in the absorption and fluorescence spectra of the dosimeter material after reaction with ammonia and/or amines is shown in FIG. 8.

The qualitative and/or quantitative detection of ammonia and/or amines, in particular in a gas mixture, can, according to the invention, be carried out by a method comprising the following steps:
a) providing a dosimeter material according to the invention;
b) interaction of the ammonia and/or amines with the dosimeter material;
c) measurement of a fluorescence property and/or absorption property of at least one section of the dosimeter material.

Quantification via the absorbance properties of the indicator can be performed over the range between 490 and 530 nm or over the range of 400 to 450 nm (FIG. 8A).

Fluorescence, unlike absorption, is free of background, and changes in the fluorescence spectrum can be measured much more sensitively. In the fluorescence spectrum, excitation in the wavelength range between 400 and 450 nm or multiphoton excitation in the range of 700 to 800 nm results in a significant change in the maxima at 600 nm, 650 nm and 720 nm (FIG. 8 B). For exact quantification, the ratio of two of these maxima can be determined.

The reaction of the dosimeter material with amines also leads to a significant change in fluorescence lifetime. FIG. 9 on the left side shows images with color coding for the fluorescence lifetime of the dosimeter material (A: green; B: red). The fluorescence lifetime measurements were performed with the multiphoton microscope with time-correlated single photon detection. The black background consists of the immobilization matrix, the embedded indicator appears as particles with a size of 20 to 90 nm. On the right side the quantitative evaluation of the color coding is shown. In parallel to the color change from green to red, the fluorescence lifetime after reaction of the indicator with amines significantly increases from 1100-1300 ps to 1600-1800 ps.

Decarboxylation products of amino acids are designated biogenic amines. Biogenic amines are ubiquitously present in food in low concentrations. Above certain concentrations, biogenic amines can negatively affect human health, causing pharmacological, physiological and toxic effects. Their quantities often increase as a result of the use of raw materials of inferior quality, during controlled or spontaneous microbial fermentation, or in the course of food spoilage. Particularly affected are foods such as fish, meat and sausages, cheese, wine, beer, sauerkraut, soy sauce and yeast extract. For this reason, biogenic amines are particularly suitable as chemical indicators of the hygienic quality and freshness of selected foods that are associated with fermentation or degradation to a certain extent.

Compared to the state of the art, the dosimeter material according to the invention offers the great advantage of direct detection of potentially harmful amines. The dosimeter material can directly indicate released ammonia and/or amines by changing color, absorption and fluorescence properties. These changes correlate with a quantifiable change (increase) in the ammonia and/or amine concentration, so that the condition of the samples to be examined, especially of biological test materials, can be continuously and prospectively monitored.

In principle, the dosimeter material in accordance with the invention can be used to pursue all issues in which ammonia and/or amines are released in the sense of spoilage, ageing or maturation. In the field of food this applies to all products of animal origin, since after slaughter or product processing, sustainable degradation processes begin, which, after a certain point, influence the consumption of the food. In other cases, increased amine formation also indicates a ripening process that can be positively evaluated (e.g. in cheese ripening or the production of pickled herring). In the latter cases, the dosimeter material can also be used as a ripening indicator.

Food packaging is generally not permeable to odorous and aromatic substances. The dosimeter material can be applied to the inside of the packaging so that the color change can only be caused by the amines from the respective material in the packaging, and not from any amines in the atmosphere. For this purpose, the packaging film should be impermeable to amines, and the indicator can be separated from the packaged goods with an amine-permeable film. Sandwich films could also be used.

The color change from green to red, which is already detectable with traces of ammonia and/or amines, is particularly well suited for use in the area of intelligent food packaging. In an intelligent food packaging, the shelf-life of food can be directly read from the dosimeter material, from transport via distributors to the end consumer (green=fresh; red=no longer fresh, first signs of spoilage). This approach stands out due to the simple, mobile detection of ammonia and/or amines without the need for complex laboratory analysis.

Furthermore, a fast, sensitive automated evaluation of the shelf-life of food is possible. By detecting fluorescence or absorption, the dosimeter material can be used in a wide variety of applications.

The detection of ammonia and/or amines can therefore be carried out with the dosimeter material in a simple and easy to understand manner for an end user as well as being quantifiable for the industrial user. There is currently no comparable product on the market for intelligent packaging with such a wide range of possible applications:

### 1. Dosimeter for the End User

As a carrier of food labelling, packaging is an essential source of information for consumers. It therefore has a considerable influence on the purchase decision. As a rule, the consumer has difficulty in assessing the freshness of a food product packaged in plastic film in the supermarket, because sensory analysis based on olfactory, haptic, and visual characteristics is only possible to a limited extent. For the end consumer, who needs a quick statement about the shelf-life of the desired food when shopping, most of the assessment methods currently on the market or newly developed are not suitable, because they are too complicated and expensive or contain colorants that are harmful to health. A shelf-life dosimeter must be very inexpensive to produce and must also have a signal effect that is easy to recognize (preferably like a traffic light: green=>good condition; red=>the food has changed). When buying, for example, packaged fish or meat, the end consumer is directly informed about the freshness of the product by the color change of the dosimeter material in the packaging.

### 2. Dosimeter for Food Processing Companies and the Food Retail Trade

The dosimeter material is also interesting for food processing companies to check the shelf-life of the packaged food. In this case the dosimeter material does not necessarily have to be visible to the end user. With the dosimeter material a continuous control during the whole production and transport process could be ensured, because the color change can be analyzed and quantified automatically. With an automated online control, the hitherto usual random inspection could be replaced by a quick check of each individual package. The food producer could also advertise the safety of his product with such a freshness indicator, since even the repackaging of the product, which is common in the industry, cannot manipulate the dosimeter material.

In food retail, it would also be possible to quantitatively assess the (daily) freshness, for example by measuring the fluorescence intensity with an appropriate measuring device. This could also help to ensure that fewer foods are disposed of at times when a questionable shelf-life has not yet been reached.

### 3. Other Applications

The dosimeter material can also be used in medical analysis. If a porphyrin-based dosimeter is adjusted to the desired sensitivity and its temporal response behavior is modified, it can also be used in the medical sector, for example in clinical diagnostics. For example, the amines di- and trimethylamine play an important role in the respiratory gas analysis of kidney failure. This requires very sensitive detection in the ppm range. The dosimeter material can be used here in the form of test strips or integrated into a breathing air bag to improve the ability of the exhaled amines to react with the film. Other medical indications associated with the formation of amines, e.g. in dentistry, are also conceivable. Due to the increased sensitivity and the possibility of quantitative evaluation, an application in wound healing bandages (“smart bandage”) is also conceivable.

Such a sensor can also be used in the field of environmental analysis, e.g. for water and soil protection.

The dosimeter material according to the invention and the corresponding method for the detection of ammonia and/or amines offer numerous advantages:
1. The substances produced during spoilage (or ripening), namely ammonia and/or amines, are detected directly, without a detour, such as for example by determining the pH.
2. The detection is preferably performed in the gas phase, so the dosimeter material does not necessarily have to come into contact with the food, which allows its use in a wide variety of packaging types.
3. The detection of ammonia and/or amines is very sensitive and selective, even traces of biogenic amines with low volatility such as cadaverine and putrescine can be detected.
4. The changes in absorption and fluorescence of the indicator are much more pronounced than with other known amine indicators. Especially in the case of fluorescence, very characteristic fluorescence maxima are formed. But the color change from green to red, which can be recognized visually, is also clearly visible and interpretable.
5. The indicator reacts with ammonia and/or amines in an irreversible reaction, so that “re-coloring” is not possible This makes any manipulation such as repackaging on the way to the end user more difficult.
6. The dosimeter material stands out because of its harmlessness to health compared to other known indicators for ammonia and/or amines; neither toxic nor carcinogenic effects are known. The individual components, porphyrin (e.g. also TPP and TPP-P(V)Cl₃), silica gel and polymers are considered harmless to health.
7. The dosimeter material can be produced at low cost, which would make a disposable dosimeter possible, which is especially interesting as a shelf-life indicator for food packaging

The invention is described in more detail according to the following examples.

### EXAMPLE 1: SYNTHESIS OF MESO-TETRAPHENYLPORPHYRIN (TPP)

100 g benzaldehyde are added to 1.5 l propionic acid and brought to boiling point. After carefully adding 63 g of pyrrole, heat is applied for a further 2 h under reflux cooling. After cooling and crystallization of the porphyrin, the suspension is filtered. The violet filter cake is first washed with propionic acid and then with methanol. Then it is dried at approx. 120° C. until the weight remains constant. Yield: 28.3 g (19.6% of theory).

### EXAMPLE 2: SYNTHESIS OF DICHLORO-PHOSPHORUS-(V)-TETRAPHENYLPORPHYRIN-CHORIDE

8 g of TPP are added to 200 ml of pyridine dried through a molecular sieve under argon as a protective gas. 40 ml of a 1:1 mixture of phosphorus trichloride and phosphoryl chloride are carefully added by dropping, and the dark red solution is heated to the boiling point for 4 hours under reflux. After the reaction is complete, the solution, now dark green, is concentrated to dryness on a rotary evaporator. The residue is taken up in a small amount of dry DCM and first purified chromatographically with hexane/DCM 1:2 over aluminum oxide. The product can be eluted from the column after separation of the impurities with DCM mixed with approx. 1% ethanol. After evaporation of the solvent on a rotary evaporator, the pure phosphorus porphyrin is now available. Yield: 7.9 g (64.7% of theory).

### EXAMPLE 3: PRODUCTION OF THE TPP-T(V)Cl₃ INDICATOR

20 g silica gel 60 (0.040-0.063 mm, for column chromatography) are pulverized as well as possible in an agate mortar. After drying for 24 h at 120° C., the silica gel is then stirred into a solution of 200 mg TPP-P(V)Cl₃ in 60 ml dried DCM. After distilling off the solvent on a rotary evaporator, the green indicator powder is activated at 120° C. for 24 h in a drying cabinet. The indicator is cooled down and stored in the desiccator under exclusion of moisture.

### EXAMPLE 4: PRODUCTION OF THE DOSIMETER MATERIAL

In a Prism laboratory extruder a granulate is produced from a mixture of 910 g LDPE and 90 g indicator powder. The temperature range for the extrusion is between 140 and 160° C. The speed of the twin screw is set to 250 rpm, resulting in a residence time of approx. 30 s. The hot plastic strand emerging from the extruder is cooled in a water bath and reduced to granules. In the next step, the green granulate is further processed into films in a Collin 75D flat film extruder. The processing temperature is between 160 and 185° C. with a residence time of approx. 3 min. The film emerging from the nozzle is cooled by rollers and brought to a thickness of 100-250 μm. A screw speed of 60-100 rpm generates a pressure of 150-200 bar in the extruder. If the indicator concentration is too high for the film, dilution is possible in the second step during film production by adding pure LDPE.

### EXAMPLE 5: REACTION OF THE DOSIMETER MATERIAL WITH AMMONIA, AMINES AND WATER

To investigate the response of the dosimeter material, ammonia and some amines were tested with both the indicator powder and the dosimeter film. For this purpose, about 10 mg of the powder or about 1 cm² of the film were added to a 10 ml sample glass with septum, and 5-100 μl from the gas phase above the respective amine were injected into the reaction vessel with a Hamilton syringe. The only difference between film and powder is the reaction time of the color reaction. The color of the powder usually changes immediately after the addition of the gas, but for the film the process is hindered by diffusion and can take some time (up to several hours). The advantage of the film is, however, that cross-sensitivity to water vapor is excluded. The film can be stored in water for several days without changing its color. Table 1 shows the boiling point and vapor pressure of selected amines and the color reaction of these amines with the indicator:

| | Boiling point at | Vapor pressure at | Color |
|---|---|---|---|
| Amine | 20° C. [° C.] | 20° C. [hPa]. | reaction |
| Ammonia solution 25% | 37.7 | 483 | +++ |
| Diethylamine | 56 | 253 | +++ |
| Trimethylamine | approx. 31 | | +++ |
| 31-23 wt % in ethanol | | | |
| Triethylamine | 90 | 69 | ++ |
| Ethanolamine | 171 | 0.5 | ++ |
| Hexylamine | 130-132 | 10.6 | ++ |
| 1,6-Diaminohexane | 199-204 | 0.25 | + |
| Cadaverine | 178-180 | | + |
| (1,5-diaminopentane) | | | |
| Putrescine | 158-160 | | + |
| (1,4-diaminobutane) | | | |
| Histamine | 167 (at 1.1 hPa) | | + |
| Triethanolamine | 360 | <0.01 | − |

+++ Color reaction after addition of small gas phase volume (amines with high vapor pressure: >100 hPa at 20° C.);
++ Color reaction after addition of medium gas phase volume (amines with medium vapor pressure: 1-100 hPa at 20° C.);
+ Color reaction after addition of large gas phase volume (amines with low vapor pressure: <1 hPa at 20° C.);
− no color reaction.

Preliminary tests with the dosimeter film with old fish or meat also showed a positive color reaction after some time.

### EXAMPLE 6: CROSS-SENSITIVITY TO OTHER SUBSTANCES THAT MAY OCCUR DURING SPOILAGE OF FOOD

Different low molecular weight compounds in high concentrations were brought into contact with both the highly sensitive indicator powder and the dosimeter film for several days. In these cross-sensitivity tests, no color change was observed with hydrogen sulfide, thiols, amino acids, alcohols, aldehydes or ketones.

### EXAMPLE 7: SIMPLIFIED OBSERVATIONS ON THE SENSITIVITY OF THE DOSIMETER MATERIAL

In the following, the amount of an amine is to be estimated which is necessary to change the dosimeter material from green to red. As a model consideration, a dosimeter film spot with an area of 1 cm² is assumed. The film thickness is 250 μm. At a density of LDPE of approx. 1 g/cm³, the dosimeter film spot has a mass of 25 mg. The indicator component contained in it, with a 3% part, is 0.75 mg powder. The indicator contains 1% of the active porphyrin component, i.e. the sensor spot contains 7.5 μg TPP-P(V)Cl₃. Since the molar mass of porphyrin is 750 g/mol, a substance quantity of 10 nmol can be calculated. If it is assumed that the porphyrin reacts 1:1 with amines for the color reaction, the dosimeter film spot must absorb approx. 10 nmol amine from the gas phase. If, for example, 1,6-diaminohexane with a vapor pressure of 0.25 hPa at 20° C.is now considered as a high vapor pressure model compound for amines, and it is assumed that the food packaging contains a gas volume of 0.25 l, with the ideal gas equation it can be calculated that approx. 2.5 μmol of this high vapor pressure amine are in the gas phase:

$$N = \frac{p * V}{R * T} = \frac{25 Pa * 0.25 l}{83 \; 14.5 [ Pa l {mol}^{- 1} {K}^{- 1} ] * 293.15 K} = 2.5 μmol$$

From these considerations, it follows that sufficient amine is present in the gas phase to induce a color change of the dosimeter film spot.

1-3. (canceled)

4. A dosimeter material for ammonia and/or amines, comprising
a) an indicator undergoing an irreversible color change in the presence of ammonia and/or amines, wherein the indicator comprises a phosphorus porphyrin activated by covalent bonding of the phosphorus to a silanol group, wherein an activation of phosphorus porphyrin having the formula porphyrin-P(V)X₃, wherein X is Cl or Br, is affected by the reaction of only one of the halogen ligands of the porphyrin-P(V)X₃ with the silanol group, and
b) an immobilization matrix for the indicator permeable to ammonia and/or amines, wherein the immobilization matrix is water-impermeable.

5. The dosimeter material according to claim 4, wherein the dosimeter material is present as granules and/or film.

6. The dosimeter material according to claim 4, wherein the immobilization matrix comprises polymers impermeable to water and permeable to ammonia and/or amines selected from the group consisting of polystyrene, low-density polyethylene, and a combination thereof.

7. The dosimeter material according to claim 4, wherein the indicator is obtainable by process wherein an activation of the porphyrin-P(V)X₃ by a covalent bond is affected by the reaction of one of the halogen ligands of the porphyrin-P(V)X₃ with the silanol group of silica gel.

8. The dosimeter material according to claim 4, wherein activation of dichloro-phosphorus-(V)-tetraphenylporphyrin-chloride by covalent bonding to silica gel occurs by the reaction of one of the chlorine ligands of the dichloro-phosphorus-(V)-tetraphenylporphyrin-chloride with a silanol group of the silica gel.

9. A process for preparation of the dosimeter material according to claim 4, comprising mixing the indicator with the immobilization matrix, wherein the process is carried out under exclusion of water.

10. A method for the detection of ammonia and/or amines in a mixture comprising
providing the dosimeter material of claim 4, interacting the mixture with the dosimeter material according to claim 4, and
measuring a fluorescence property and/or absorption property of at least one section of the dosimeter material.

11. The method according to claim 10, wherein the mixture is a food product.

12. The method according to claim 10, wherein the mixture is a gas mixture.

13. The method according to claim 12, wherein the gas mixture is a use is in food quality control or in medical applications such as respiratory gas analysis or wound dressings, preferably in food quality control.

14. The method according to claim 10, wherein the absorption property is a color change that can be detected visually.

15. The dosimeter material according to claim 4, wherein the indicator comprises a phosphorus porphyrin activated by covalent bonding to a silanol group of a substance comprising silica gel.

16. The indicator according to claim 15, wherein the substance consists of silica gel.

17. The dosimeter material according to claim 4, wherein an activation of phosphorus porphyrin having the formula dichloro-phosphorus-(V)-tetraphenylporphyrin-chloride (TPP-P(V)Cl₃), dibromo-phosphorus-(V)-tetraphenylporphyrin-bromide (TPP-P(V)Br₃), dichloro-phosphorus-(V)-tetratolylporphyrin-chloride (TTP-P(V)Cl₃), or dichloro-phosphorus-(V)-2,3,7,8,12,13,17,18-octaethylporphyrin-chloride (OEP-P(V)Cl₃) is effected by the reaction.

18. The dosimeter material according to claim 17, wherein an activation of phosphorus porphyrin having the formula dichloro-phosphorus-(V)-tetraphenylporphyrin-chloride (TPP-P(V)Cl₃) is effected by the reaction.

19. The dosimeter material according to claim 5, wherein the dosimeter material is present as film.

20. The dosimeter material according to claim 6, wherein the immobilization matrix comprises polymers impermeable to water and permeable to low-density polyethylene.

21. The process according to claim 9, wherein the mixture of indicator and immobilization matrix is prepared as granules and/or film.
